(19) United States
(12) Patent Application Publication
(10) Pub. No.: US 2011/0300197 A1
(43) Pub. Date: Dec. 8, 2011

(54) FOOD PRODUCT

(75) Inventors: Philip Martin McGenity, Berkshire (GB); Marie-Louise Baillon, Berkshire (GB); Andrew Newton, Berkshire (GB)

(73) Assignee: Mars, Incorporation, McLean, VA (US)

(21) Appl. No.: 12/993,912

(22) PCT Filed: May 28, 2009
(86) PCT No.: PCT/GB2009/001330

§ 371 Date: Feb. 15, 2011

(30) Foreign Application Priority Data
May 28, 2008 (GB) 0809665.3

(51) Int. Cl. A61K 8/11; A61Q 11/00; A61K 8/97

(52) U.S. Cl. 424/401; 424/58

(57) ABSTRACT

The present invention relates to a food product comprising an outer portion and an inner portion, wherein the inner portion comprises myrtle and has a moisture content of from about 10% to about 25% by weight, the food product for use in oral health applications, and the use of the food product in the improvement or maintenance of oral health in an animal, preferably through the reduction or control of tartar and/or dental plaque and/or alteration of the bacterial content of dental plaque, in the oral cavity of the animal. The invention also includes the food product for use in the prevention or treatment of gingivitis in an animal. The invention also provides a method for improving or maintaining oral health in an animal.

The present invention relates to a food product comprising an outer portion and an inner portion, wherein the inner portion comprises myrtle and has a moisture content of from about 10% to about 25% by weight, the food product for use in oral health applications, and the use of the food product in the improvement or maintenance of oral health in an animal, preferably through the reduction or control of tartar and/or dental plaque and/or alteration of the bacterial content of dental plaque, in the oral cavity of the animal. The invention also includes the food product for use in the prevention or treatment of gingivitis in an animal. The invention also provides a method for improving or maintaining oral health in an animal.

The need to maintain or improve oral health in an animal is of great importance. Poor oral health can lead to gum disease (gingivitis) and ultimately tooth loss, which can have severe effects on the wellbeing of the animal.

Poor oral health can be caused by a number of diseases and conditions. One of the most prevalent amongst cats and dogs is periodontal disease. Periodontal disease affects all cats and dogs at some stage during their lives. The aetiological agent in all cases of periodontal disease is plaque.

Current dietary methods for reducing or controlling plaque formation (and therefore associated conditions, such as gingivitis), in companion animals are usually mechanical means, such as hard chews or treats which act to scrape the plaque from the teeth, when chewed or consumed by the animal. The mechanical means rely on texture for their efficacy and a chewy rather than brittle texture is preferable to resist breakage of the means and therefore to also increase tooth cleaning time during chewing. Cats are less keen than dogs to chew for prolonged periods. Therefore products for various animals differ in texture to allow for these different preferences.

Textured toys may also be employed, to remove plaque mechanically from the surface of the teeth, without the animal ingesting any of the product that provides the textured surface.

However, the removal of plaque by mechanical means such as textured foodstuffs or toys relies upon the animal spending sufficient time chewing the mechanical means to scrape the plaque from the surface of the teeth. The amount of time required is difficult to assess and to monitor. In addition, plaque control on all tooth surfaces in the oral cavity is difficult to achieve via mechanical abrasion alone and certain teeth receive more efficient cleaning than others.

Plaque may also be removed or reduced by cleaning the teeth by brushing. However, owner compliance with toothbrushing is poor, with the result that very few dogs and cats receive a daily oral care regime of toothbrushing.

As an alternative to mechanical means for the removal of plaque, certain synthetic compounds such as chlorhexidine and triclosan can be used as antibacterial agents to reduce plaque. However, these compounds are broad spectrum antibacterial agents and, as such, may cause an imbalance in healthy gut microflora populations when ingested regularly. In addition, certain plaque bacteria have been associated with periodontal health and treatment with broad spectrum antibacterials would potentially kill these populations and would actually result in a less healthy oral microflora, leading to a reduction in oral health.

Accumulation of bacterial biofilms on the surface of a tooth can lead to gingivitis if not sufficiently addressed. Gingivitis is an inflammation of the gums caused by bacterial plaque that accumulates on the gum line. It can cause soreness, redness and bleeding of the gums.

An additional contributory factor to poor oral health is calculus (also known as tartar). Since calculus cannot be removed by toothbrushing in normal cases, it accumulates on the tooth surface and provides a surface on which additional plaque can readily accumulate. This is a further indication of poor or deteriorating oral health.

The addition of calculus formation inhibitors such as sodium tripolyphosphate to pet foodstuffs or human oral care products helps to prevent calculus accumulation.

However, this does not address the bacterial community composition within the dental plaque that is contributing to the detrimental effects of periodontal disease on the oral health of the animal.

Therefore, there is a need for reducing the effects of dental plaque in an animal, in particular by natural methods, without relying solely on mechanical means or synthetic chemicals or compounds and without stressing the animal. Furthermore, there remains a need for the prevention and treatment of gingivitis in an animal.

Accordingly, the present invention provides a food product comprising an outer portion and an inner portion, wherein the inner portion comprises myrtle and has a moisture content of from about 10 to about 25% by weight.

Myrtle (Myrtus communis) is a flowering plant in the family Myrtaceae, native to southern Europe and north Africa.

The inventors have unexpectedly found that myrtle, in combination with a food product that is designed to scrape plaque from the teeth of an animal, is able to improve and/or maintain oral health in an animal.

Preferably, the food product improves or maintains the oral health of the animal by controlling or reducing dental plaque in the animal, by which it is meant that disease causing factors produced by the plaque and/or dental plaque are reduced or inhibited in the oral cavity of the animal.

Dental plaque is a mixed microbial community consisting of aerobic and anaerobic bacteria. Although plaque may vary between individuals the formation process can be broken down into three key events of (i) primary colonisation (adhesion); (ii) secondary colonisation (co aggregation); and (iii) maturation (virulence).

Plaque development begins with a tooth surface covered with a film of proteins and glycoproteins called the tooth salivary pellicle. Pioneer bacterial species adhere to molecules within the salivary pellicle, first forming a monolayer and subsequently pallisades of bacteria perpendicular to the tooth surface.

The microbe is held for a brief period by a weakly attractive force, during which time a number of specific adhesion mechanisms hold the cell close to the surface for a significant time period. These specific interactions may be a combination of lectin-like, electrostatic and hydrophobic interactions that in some instances could involve delicate structures called fibrils or fimbriae that project from the cell surface. Following this, initial attachment is rendered effectively irreversible by the production of extra-cellular polymers.

In humans streptococci are the most common primary colonisers making up between 47-52% of all bacteria adhering to the salivary pellicle.

During and after this initial phase, secondary colonisation by a variety of bacteria occurs leading to a large increase in bacterial diversity. Foremost among the events of secondary colonisation is the process of coaggregation whereby the primary colonisers now act as the substrate for colonisation.

Coaggregation has been described as ‘the recognition between surface molecules on two different bacterial cell types so that a mixed cell aggregate is formed’. It has also be described as the adherence among partner cells in a suspension'.

Coaggregation is a highly specific process that takes place between specific bacterial ‘partners’. Each strain has its own set of partners and mechanisms of cell-cell recognition. Groups of strains also exist which are able to coaggregate with several other strains. Based on human studies, one such organism that dominates these later colonisers is Fusobacterium nucleatum, which is a dominant organism in mature dental plaque.

Coaggregation is known to play an important role in human plaque formation. Coaggregation between different strains of canine oral bacteria has been determined in vitro suggesting a similar role for this behaviour in dental plaque formation and development in other animals.

At some point during the development of the plaque biofilm, the rate of change in the overall composition slows. The point at which this happens is currently unknown, although it is thought to take several days for the biofilm to reach this state.

In human plaque, a succession of bacterial species occurs as Gram-positive cocci and rods are progressively replaced by Gram-negative filamentous and flagellated organisms. The maturing biofilm also tends to become increasingly anaerobic as it increases in depth.

It is at this point that the biofilm can be said to have reached a climax community, where a number of the bacteria are reliant on others within the biofilm for their survival. It is during this phase that many organisms associated with periodontal disease are present. These bacteria produce a number of compounds that are the causative factor of periodontal disease, such as proteases and haemolysins. Proteases, in particular trypsin, are reported to have a host of abilities, including the ability to degrade immunoglobulins, inactivate cytokines and their receptors, degrade host tissues and promote bleeding in the oral cavity. The bacteria of the plaque is known as the plaque biomass.

Pathogenic bacteria, such as Peptostreptococcus are often present in dental plaque, as well as black pigmenting anaerobes, such as Porphyromonas, Bacteroides and Prevotella, all of which are thought to contribute to disease states.

The food product of the invention is useful for inhibiting the formation of such biofilms and/or inhibiting the detrimental activities of the biofilm and therefore improving or maintaining oral health by controlling or reducing dental plaque and tartar in an animal. The food product of the invention is also provided for the prevention or treatment of gingivitis in an animal.

By reducing the level of pathogenic bacteria in the biofilm, the health of the dental plaque is improved. Thus, the foodstuff product of the invention is useful in altering the bacterial content of the plaque, preferably by reducing the pathogenic bacterial content of the plaque in the oral cavity of an animal. The food product may also promote the healthy bacteria of the plaque. The food product of the invention is useful in improving the health of the dental plaque present in the oral cavity of an animal.

The food product of the invention preferably reduces the level of inflammatory proteases and/or black pigmenting anaerobes in dental plaque in an animal. These are key disease causing agents that are found in dental plaque.

Most preferably, food product inhibits or reduces pathogenic bacteria in dental plaque, which preferably includes Peptostreptococcus sp.

The food product of the invention is suitable for any animal including a human. However, in a preferred embodiment the animal is a companion animal. By companion animal it is meant any animal that is kept as a pet, which includes a cat, a dog, a horse, a rabbit, or a guinea pig. Preferably, the food product is for a cat or a dog.

The myrtle variety is preferably Myrtus communis, which is also known by several other names including Myrtus baetica, Myrtus italica, Myrtus romanifolia, Myrtus macrofilia, Myrtus littoralis, Myrtus minima. The skilled person understands that other names are used to refer to this species of myrtle including Myrtus baetica var. vidalii, Myrtus communis var. christinae, Myrtus communis var. eusebii, Myrtus communis var. gervasii, Myrtus italica var. briquetii, Myrtus italica var. petriludovici, Myrtus communis var. acutifolia, Myrtus communis var. angustifolia, Myrtus communis var. baetica, Myrtus communis var. belgica, Myrtus communis var. mucronate, Myrtus communis var. romana, Myrtus major Garsault, Myrtus minor Garsault, Myrtus acuta Mill, Myrtus baetica Mill, Myrtus belgica Mill, Myrtus italica Mill, Myrtus minima Mill, Myrtus littoralis Salisb, Myrtus macrophylla, Myrtus microphylla Myrtus romanifolia, Myrtus communis subsp. Mucronata, Myrtus media, Myrtus romana Hoffmanns, Myrtus angustifolia, Myrtus buxifolia Raf, Myrtus lanceolata Raf, Myrtus latifolia Raf, Myrtus oerstedeana, Myrtus sparsifolia, Myrtus veneris Bubani, Myrtus communis var. acuminate, Myrtus communis var. italica (Mill.), Myrtus communis var. lusitanica, Myrtus borbonis Sennen, Myrtus acutifolia (L.), Myrtus augustinii, Myrtus baui, Myrtus briquetii, Myrtus christinae, Myrtus communis var. balearica, Myrtus communis var. foucaudii, Myrtus communis var. grandifolia, Myrtus communis var. joussetii, Myrtus communis var. neapolitana, Myrtus eusebii, Myrtus gervasii, Myrtus josephi, Myrtus mirifolia, Myrtus petri-ludovici, Myrtus rodesi, Myrtus theodori, and Myrtus vidalii

The myrtle contained within the inner portion of the food product (and optionally in the outer portion) of the invention can be the whole plant or part thereof. It may be the root, bark, stem, leaf, sap, flower or any combination thereof. The myrtle may be dried, crushed, ground or shredded. Preferably, the myrtle to be used is myrtle leaf. Preferably, the myrtle leaf is in the form of a dried powder.

Additionally or alternatively an extract of myrtle may be used. Suitable extracts include methanol extract, ethanol extract, chloroform extract or water extract. Any other suitable extract may be used, as understood by the skilled person.

The myrtle may comprise between 0.1%-20% by weight of the food product, more preferably 1-15% by weight, more preferably 3-10% by weight, or 1, 2, 3, 4, 5, 6, 7, 8, 9, or 10% by weight. Preferably, the myrtle comprises about 3% by weight of the food product. Most preferably, the myrtle comprises about 1.5-2.0% by weight of the food product. The myrtle comprises about 3 to 8% of the inner portion, more preferably about 4% of the inner portion.

The food product may comprise myrtle as the only active ingredient with respect to the improvement or maintenance of oral health. Alternatively, the food product may comprise myrtle as part of a cocktail including one or more further oral health improving or maintaining, or plaque reducing or controlling components. Such components may include zinc sulphate or sodium tripolyphosphate. Additional natural active ingredients may also be included.

Hereinafter in this text, the term food product covers all compositions that come into contact with the oral cavity, preferably the surface of a tooth of an animal, including a foodstuff, diet and supplement. Any of these forms may be solid, semi-solid or liquid. The food product may comprise a paste, a gel or a gel-like composition. Preferably, the food product comprises a hard textured outer portion (shell) and a softer inner portion (core), which may be in the form of a paste or a gel. The food product is preferably a supplement to the main diet of the animal. The inner portion may be of similar texture and material to the outer portion. The supplement may be used every day i.e. seven times per week, or less frequently, for example, 2 times per week. Alternatively, the supplement may be used, 6, 5, 4, 3 times or once per week or even twice per day.

By hard outer portion it is meant that the hardness is sufficient to require chewing by the animal in order to consume the product. The hardness is sufficient to “scrape” on the tooth's surface while the animal is chewing, in order to remove a proportion of any plaque that is present on the tooth's surface. The outer portion may be toughened with the addition of particular starches in order that the food product must be chewed continuously in order to consume it. This increases the benefits of the food product by prolonging the chewing action and the exposure to the myrtle.

The food product may be used to supplement any foodstuff that does not contain sufficient levels of myrtle to improve or maintain oral health including prevention or treatment of gingivitis, or to control or reduce dental plaque in an animal, by way of reduction or inhibition of disease causing factors and/or biomass in the plaque.

The concentration of myrtle in the food product may be used in addition to the animal's main diet or foodstuff. This can be done by including a quantity of the supplement with the animal's diet or by additionally feeding the animal a quantity of the supplement.

Preferably the myrtle or a composition comprising myrtle is incorporated into a commercial petfood product composition or a commercial dietary supplement composition. The petfood product may be a dry, semi-dry, a moist or a liquid (drink) product. Moist products include food which is sold in tins or foil containers and has a moisture content of 70 to 90%. Dry products include food which have a similar composition, but with 5 to 15% moisture and presented as biscuit-like kibbles. The food product of the invention is preferably packaged. In this way the consumer is able to identify, from the packaging, the ingredients in the food and identify that it is suitable for the animal in question. The packaging may be metal (usually in the form of a tin or flexifoil), plastic, paper or card. The amount of moisture in any product may influence the type of packaging which can be used or is required.

The food product according to the present invention may comprise, as one or more of the components any product which an animal may consume in its diet. Thus, the invention covers standard food products for humans or other animals, as well as pet food snacks (for example snack bars, biscuits and sweet products). The food product may be a cooked product. It may incorporate meat or animal derived material (such as beef, chicken, turkey, lamb, blood plasma, marrowbone etc, or two or more thereof). The food product alternatively may be meat free (preferably including a meat substitute such as soya, maize gluten or a soya product) in order to provide a protein source. The food product may contain additional protein sources such as soya protein concentrate, milk proteins, gluten etc. The food product may also contain a starch source such as one or more grains (e.g. wheat, corn, rice, oats, barely etc) or may be starch free. A typical dry commercial dog and cat food contains about 30% crude protein, about 10-20% fat and the remainder being carbohydrate, including dietary fibre and ash. A typical wet, or moist product contains (on a dry matter basis) about 40% fat, 50% protein and the remainder being fibre and ash. The food product of the present invention is particularly relevant for a foodstuff as herein described which is sold as a foodstuff or supplement for a cat or a dog.

In the present text the terms “domestic” dog and “domestic” cat mean dogs and cats, in particular Felis domesticus and Canis domesticus.

The food product is preferably in the form of a chew or treat which the animal may consume in addition to a main meal foodstuff. The myrtle may be provided as a coating on or incorporated within the food product. The myrtle is present within the inner portion or core. The myrtle may also be present in the outer portion, either within the composition of the outer portion, or applied as a coating.

The inner portion, the core, is a composition having a moisture content of from about 10% to about 25%. The moisture content may be 10%, 12%, 15%, 20%, 22% or 25% by weight. The water activity is preferably below 0.8, more preferably below 0.7, most preferably below 0.6. The moisture content of the product as a whole is preferably from 10% to 25%. Most preferably, both the inner portion and the outer portion each have a moisture content of 10% to 15%, most preferably of about 13% to 14%.

The inner portion may be in the form of a gel or a paste or a gel-like matrix. By gel it is meant a matrix consisting of a gelling material (such as a polymer or a fine particulate material) dispersed in a medium (such as water) in such a way as to produce a semi-solid material with a jelly-like texture. Gel-like can mean having the appearance and/or texture of a gel, but a lower moisture content than a gel. A gel-like substance may be transparent, semi-opaque, or opaque.

Alternatively, the inner portion may be an aerated starch based material similar to the outer portion. Such an inner portion is tough (i.e. not brittle or easily bitten off) but compressible due to the presence of starch and the air pockets introduced by aeration during manufacture. In such a product the inner portion is substantially the same in texture as the outer portion, but has a different composition. The composition may differ in its content of myrtle and/or protein and other ingredients such as palatants.

The inner portion may be coloured with natural colourings and/or those known to the skilled person for use in food products.

The inner portion may be in the form of a paste. By paste it is meant a viscous composition that retains its position within the outer portion but is soft and preferably will coat (at least transiently) the teeth of the animal when consumed. The paste may be of a similar consistency to, for example, toothpaste.

In a preferred embodiment, the food product resembles a bone, in which the outer portion comprises one or more starches, palatants, flavourings and water, and the inner components comprises one or more starches, palatants, myrtle and water. Suitable starches include maize starch, potato starch or other cereal starch. Palatants and flavourings may include meat flavours, such as beef liver powder, chicken smoke flavour, fish flavour or fats. The outer portion may optionally comprise one or more stabilisers or preservatives, and may also optionally comprise myrtle. The outer portion is in the form of a tube. The cross section of food product is preferably approximately triangular. The sides of the triangle may be curved, concave, or convex. The triangle may be asymmetric.

The skilled person will appreciate that any cross section shape may be used for the tube. Preferably the cross section of the product is a shape that has sharp corners (having an internal angle of less than 90°) such as a triangle, or diamond, in order to provide edges that can mechanically remove the plaque from the tooth's surface. The shape may also be chosen to give the product strength, particularly the outer portion. This results in a food product that requires prolonged biting or chewing action in order to break it down. This means that both the abrasive action and exposure to active ingredients, including myrtle, is increased.

The edges of the food product may be twisted around the length (i.e. the longitudinal axis) of the food product to provide improved plaque removal by gentle abrasion.

The ends of the tube may be sealed so that the inner portion is not visible. Alternatively, the ends may be open and the inner portion may be exposed. Each individual product may vary in size, depending on for which animal it is intended. For example, a small dog or a cat will require a smaller product than a large dog will require. The product may vary from 40 g to 200 g, preferably the product is about 60 g, about 100 g or about 160 g. The length of the product is preferably from about 100 mm to 200 mm Preferably the product is about 100 to 200 mm, 121 to 140 mm or 141 to 160 mm, most preferably about 110 mm, about 130 mm or about 150 mm in length. The diameter of the cross section is preferably from 30 mm to 50 mm, most preferably about 35 mm, about 40 mm or about 45 mm.

The twist along the longitudinal axis may be from 100 to 150°. The twist ensures slower consumption of the food product by the animal as the shape is awkward to bite and must be chewed. This increases the time that the mechanical action of the food product acts on the tooth's surface and also increases the exposure of the teeth, saliva and gums to the myrtle and any other active ingredients.

The inner portion may comprise from about 10% to 75% of the finished product. The myrtle may comprise from 1% to 10% of the finished product. The inner portion is preferably a starch matrix having a low protein content. The inner portion is preferably tough and difficult to break and therefore requires prolonged chewing.

However, it is also preferably compressible and will “give” when pressure is applied by the jaws of the animal.

In an alternative preferred embodiment, the outer portion may be a tube structure, with a textured outer surface achieved by a shaped cross section, such as a star-like shape with sharp corners having an internal angle of less than 90°. The star-like shape may have 3, 4, 5, 6, 7, 8, 9 or 10 points. These corners are more likely to reach into the interdental spaces to mechanically remove plaque and also to allow the myrtle and other active ingredients contact with the tooth's surface in these areas of the mouth of an animal. The edges of the outer portion (resulting from the points of the star-shaped cross section) may twist around the longitudinal axis of the outside surface of the tube to provide increased abrasion on the surface of the tooth when consumed. The twist in this embodiment is preferably from 70 to 100°. The outer portion may comprise starches, palatants, flavourings and water, as set out above. Additionally, the outer portion may include a plant fibres for structure, such as hemp. The inner portion of this preferred embodiment comprises myrtle, modified starch and fructose and/or glucose syrup. Preservatives and stabilisers may be present also. Preferably, the inner portion of this preferred embodiment contains no protein.

In this embodiment the product may be from 15 g to 45 g in weight. As mentioned previously, the food product may be of different sizes depending on the animal to which it is to be fed. Therefore, the food product may be about 18 g, about 30 g or about 42 g. The food product may be between 75 mm and 175 mm in length. Preferably the product is from 85 mm to 95 mm, from 125 to 135 mm or 145 to 155 mm in length, most preferably about 90 mm, about 130 mm or about 150 mm in length. The cross section of the food product may be from 10 mm×12 mm to 20 mm×25 mm. Preferably, the cross section of the product is about 12.5 mm×15.5 mm, 14 mm×18.5 mm or 16 mm×20.5 mm.

The food product of the invention may be produced by co-extrusion, wherein the outer portion and inner portion are extruded together, with the outer portion extruded from an annular nozzle surrounding an inner nozzle from which the inner portion is extruded. Alternatively, the outer portion may be extruded as an empty tube, and then filled separately with the inner portion. In this case the inner portion is prepared in a mixer then pumped into the extruder.

The food product of the invention has the benefit of improving or maintaining the oral health of the animal by removing plaque through the mechanical (gentle abrasive) action of the product against the surface of the teeth of the animal, as well as by the action of the myrtle in the inner portion, (and optionally in the outer portion).

The inhibition of certain plaque biofilm forming bacteria by myrtle results in the control or reduction of dental plaque in an animal by the reduction of the bacterial content of the dental plaque.

The food product of the invention may be used for an animal with any level of oral health in order to improve or maintain oral health in the animal.

The food product may be used for an animal with good or acceptable oral health in order to maintain oral health. The composition in this case may control dental plaque formation and minimise the destructive effects of certain plaque bacteria on the periodontal health of the animal.

In cases of moderate to severe periodontal disease, the animal may require veterinary and/or dental attention prior to using the food product in order to achieve oral health benefits and reduce the frequency of future veterinary and/or dental intervention.

The food product is consumed and during use the oral cavity of the animal is exposed to the myrtle of the food product, and preferably the composition has direct contact with the surface of a tooth of the animal. Most preferably, the surface of a tooth is directly contacted with the myrtle of the inner portion of the food product, as well as being contacted by the outer portion to physically remove a proportion of the plaque.

A second aspect of the invention provides the food product of the first aspect for use in the improvement or maintenance of oral health in an animal. Preferably, the oral health is improved or maintained by the control or reduction of tartar and dental plaque in the animal including reduction and/or inhibition of disease causing factors, biomass or pathogenic bacteria. The food product for use in the prevention or treatment of gingivitis is also provided.

The invention, as a third aspect, also provides a method for the improvement or maintenance of oral health in an animal comprising administering to the animal a food product of the first aspect. Preferably, the method improves or maintains the oral health of the animal by the reduction or control of tartar and dental plaque in the animal, as previously defined.

In the method of the third aspect, the oral cavity of the animal is exposed to the composition, by way of consumption of the food as a supplement to the main diet of the animal. The method may be carried out, daily, weekly or monthly. Preferably the method is carried out 2, 3, 4, 5, 6 or 7 times a week.

Preferably, the method is for use in an animal susceptible to poor oral health or dental plaque, gingivitis or periodontal disease.

The food product may be administered to an animal with poor oral health to reduce the amount of dental plaque or factors contained therein, and then continued feedings may be carried out to control, reduce or inhibit the formation of further tartar or dental plaque or any one or more of the factors contained therein. The animal may require veterinary and/or dental treatment before or during use of the food product to remove calculus deposits (tartar) in order to see a beneficial effect of the food product.

By poor oral health is meant the presence of a number of indicators of this status including calculus and plaque accumulation, gingivitis, oral malodour, presence of gingival recession and/or periodontal pockets, as will be appreciated by the skilled person.

All features of each aspect of the invention relate to all other aspects mutatis mutandis, as appreciated by the skilled person.

The invention will now be described with reference to the following non-limiting examples and figures, in which:

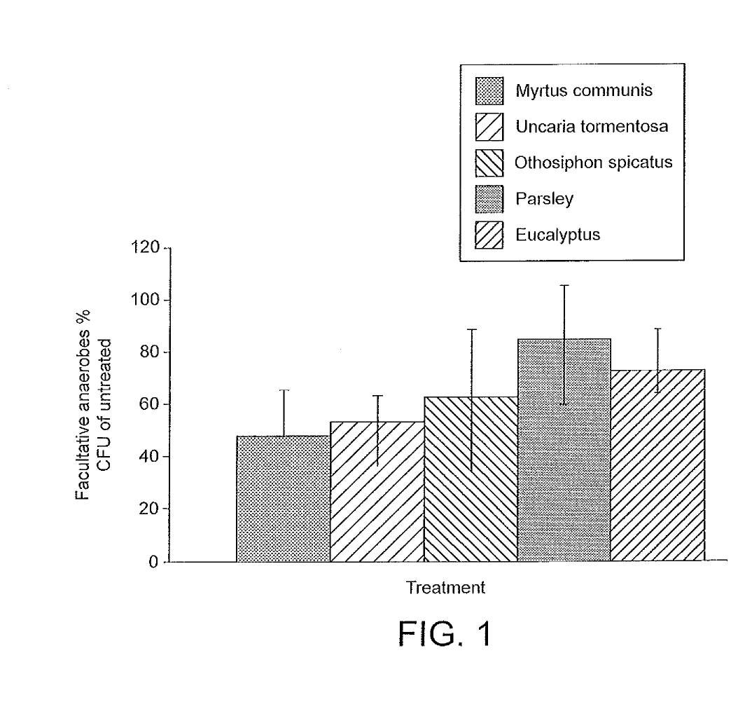

FIG. 1 shows the effect of myrtle on facultative anaerobes cultured from treated biofilms expressed as a percentage of untreated controls. Untreated CFU (100%)=4.05×10̂7/ml;

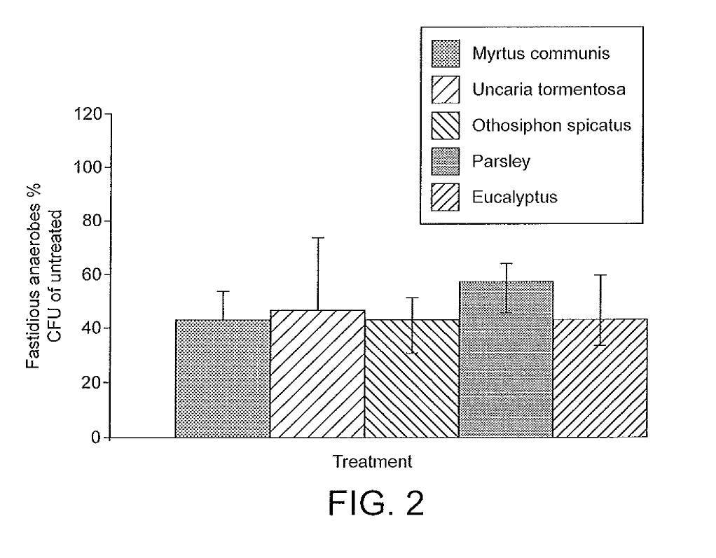

FIG. 2 shows the effect of myrtle on fastidious anaerobes cultured from treated biofilms expressed as a percentage of untreated controls. Untreated CFU (100%)=2.96×10̂7;

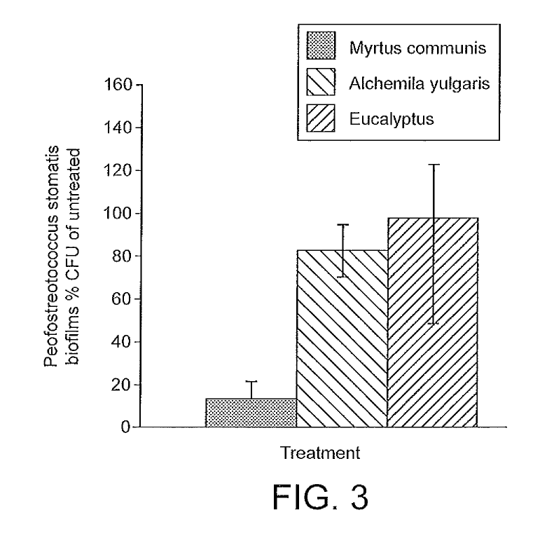

FIG. 3 shows the effect of myrtle on Peptostreptococcus stomatis colonies cultured from treated single species biofilms expressed as a percentage of untreated controls. Untreated CFU (100%)=1.34×10̂7;

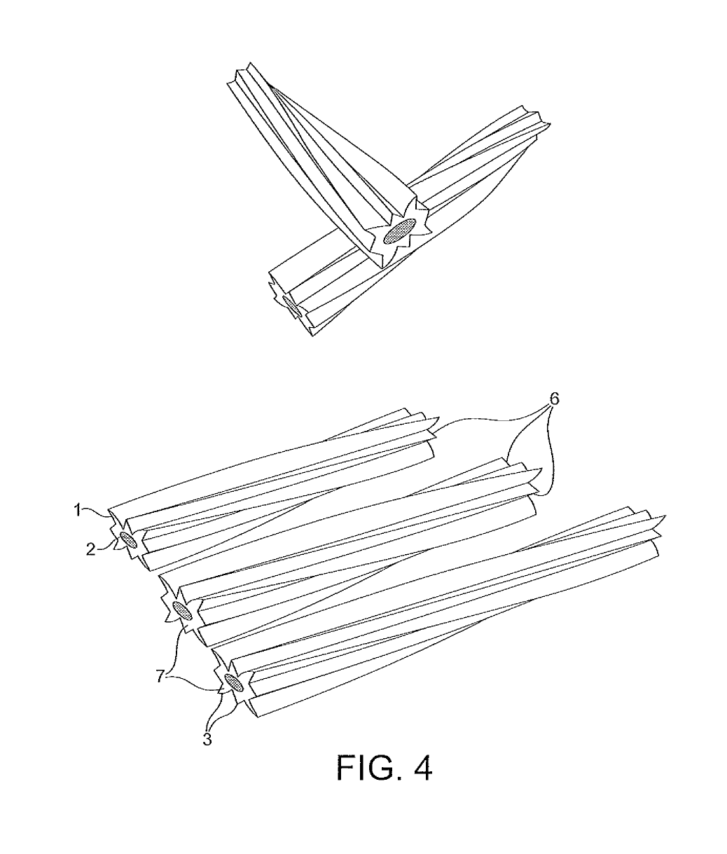

FIG. 4 shows an embodiment of the food product of the invention in which the outer portion (1) is roughly star shaped in cross section and the inner portion (2) forms an approximate oval shape. The points (3) of the cross section form ridges (6) which twist around the product along its length. The ends (7) of the product are open; and

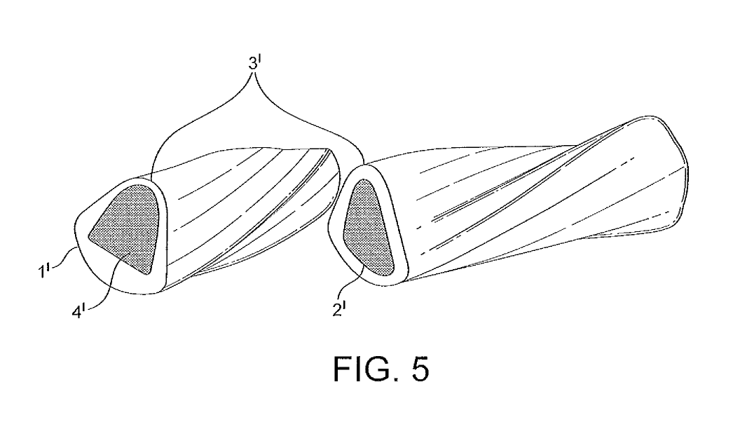

FIG. 5 shows an alternative embodiment of the invention in which the outer portion (1′) is approximately triangular in cross section, and the inner portion (2′) is similar in shape. The corners (3′) of the triangular shape twist along the longitudinal axis of the product, along it's length. The ends (4′) of the product are not sealed, meaning that the inner portion is visible.

### EXAMPLES

Myrtle was tested for its ability to control or reduce dental plaque in an animal by way of the following in vitro experiments. Supragingival plaque was obtained from dogs and various assays were carried out, as described below, to determine whether myrtle has the ability to improve or maintain oral health in an animal.

### Example 1

Initial assays were set up to determine whether myrtle is suitable for use in an animal for improving or maintaining oral health.

These assays include the ability to inhibit adhesion of plaque forming bacteria, inhibit growth of oral bacteria, inhibit protease production in oral bacteria and inhibit haemolysis caused by oral bacterial strains.

Myrtle inhibited adhesion by up to 100%, growth by up to 93%, protease production by up to 57% and showed the ability to inhibit haemolysis in 5 out of 8 oral bacterial strains tested.

These results showed that myrtle has the ability to inhibit undesirable oral bacteria and therefore it was tested in further assays for its ability to maintain or improve oral health in an animal.

### Example 2

Assay Inoculum: Plaque and Saliva Sampling from Dogs

The assay requires fresh supragingival canine dental plaque and saliva for inoculation. The inoculum consists of pooled dental plaque and unfiltered saliva sampled from a group of 14 dogs, varying in age, breed and oral health status.

The plaque and saliva were resuspended in artificial saliva to form the inoculum of approximately 15% plaque and 30% saliva.

### Assay Set-Up

The plate biofilm assay (PBA) utilises a 24 well plate format in which biofilms, representative of canine dental plaque, are grown on hydroxyapatite (HA) discs. Prior to being introduced to the 24 well assay plate, each HA disc is preconditioned for 2 hours in a solution of 50% filter sterilised canine saliva in artificial canine saliva. The preconditioning step stimulates the formation of a salivary pellicle on the HA disc surface. Following preconditioning, each HA disc is placed individually into a well on the 24 well plate. The inoculum is divided into two equal aliquots and the active added to one aliquot at the appropriate concentration. The other aliquot represents the control (no active). A 1 ml inoculum is added to each well and the assay plate incubated aerobically with shaking at 38° C. for 48 hours. After 24 hours and 30 hours, the discs are transferred into fresh artificial saliva containing the active at the appropriate concentration as before. Biofilm-covered HA discs are removed from the assay plate for analysis after 48 hours. Each HA disc, with the exception of those being used for biomass quantification, is placed into 500 μl PBS and vortex mixed for 30 seconds to remove biofilm growth from the disc into solution. Biofilm suspensions are then used for analysis. Biofilm-covered HA discs that are being used for biomass quantification are removed from the 24 well assay plate and used directly in the crystal violet assay.

### Example 3

### Myrtle Extracts Tested in the PBA

A methanol extract of myrtle was used for testing in the canine PBA. Extractions were performed as described previously.

The raw botanical of myrtle leaf was tested against clove (dried flower buds), parsley (leaf) and eucalyptus (leaf) in the canine PBA at 500 μg/ml and 5000 μg/ml. Myrtle shows an improved performance over parsley and eucalyptus in protease and biomass inhibition at both 500 μg/ml and 5000 μg/ml. Myrtle performs as well as clove in black pigmenting colony and protease inhibition at 5000 μg/ml.

In addition, chlorhexidine (Lloyds Pharmacy) was included as the gold standard reference or positive control. However, chlorhexidine is undesirable for use in animal compositions since it is a synthetic chemical and may have potential toxic effects as it is a chemical used in its purest form.

### Example 4

### Biofilm Measures

The following analyses were used to assess the biofilms produced in the canine PBA and the effects of myrtle and the non-botanical compounds on biofilm development:

Biomass quantification (crystal violet assay)
Protease activity
Bacterial viable counts

A brief description of each assay is given below.

### Biomass

The total amount of biofilm grown on the HA discs was quantified using the crystal violet staining method. Biomass was represented as being directly proportional to the OD reading at 595 nm (0D₅₉₅) of the samples compared to controls. Results were expressed as the reduction in OD₅₉₅ seen in active-treated samples compared to no active controls, reflecting the effect of the active treatment on the amount of biofilm growth on the disc.

Myrtle reduced biomass by 59.5%.

### Protease Activity

Trypsin-like protease activity was measured using the liquid BAPNA assay, a colourimetric assay in which the amount of trypsin present in a sample is directly proportional to the intensity of the colour developed. Samples were quantified against a trypsin standard curve and results expressed as the percentage inhibition of protease activity in active-treated samples compared to controls.

Myrtle reduced protease production by 74.34%

### Bacterial Counts

Viable numbers of bacteria were quantified using Columbia blood agar plates supplemented with haemin and menadione. Aerobes were counted after incubation for 2 days and anaerobes, including black pigmenting colonies (BPC), were counted after incubation at appropriate conditions for 9 days. Plate counts are expressed as colony forming units (cfu) per ml and differences between control and active plates are expressed in logs.

Myrtle reduced plate counts of black pigmenting bacterial colonies by 3.75 logs, compared to controls. This particular group of bacteria are thought to be important in periodontal disease.

### Example 5

### Statistical Analysis of Data

Each sample was repeated 5 times within the assay. Unless otherwise stated, all extracts were tested in the assay at a concentration of 500 μg/ml. For each sample, all of the values obtained were logged and the means calculated from the log values.

A 2-tailed t-test with unequal variance was then performed. An unequal variance analysis was selected as the individual analyses were independent i.e. the measures were not comparable to one another. For each data set, p values were obtained and these gave an indication of the reproducibility of the data.

### Results

A table summarising how myrtle performed in the tests is set out below:

TABLE 1

| | Aerobe | Anaerobe | BPC | Protease | Biomass |
|---|---|---|---|---|---|
| Common | (Log 10 | (Log 10 | (Log 10 | (% re- | (% re- |
| name | reduction) | reduction) | reduction) | duction) | duction) |
| Chlorhexidine | 2.87 | 2.48 | 2.74 | 95.76 | 94.40 |
| Myrtle leaf | 0.05 | −0.10 | 3.75 | 75.34 | 59.50 |
| Orthosiphon | −0.09 | 0.02 | 2.59 | 24.53 | 14.60 |
| Tepezcohuite | 0.25 | −0.42 | −0.51 | 80.25 | −27.40 |

As can be seen, Myrtus communis significantly reduced black pigmenting colony counts and had a significant inhibitory effect on protease and biomass.

### Example 6

### Testing of Raw Material

The raw plant material of myrtle was also tested in the Plate Biofilm Assay, as well as the extracts described above. The raw plant material was prepared through a 250 μm pore size sieve and was tested at 5000 μg/ml in the assay. The raw material was as effective at inhibiting biofilm formation as the previously tested extracts.

### Example 7

### Inhibition of Human Plaque

Myrtle leaf powder was tested for inhibition of biofilm formation in a human form of the Plate Biofilm Assay. The final concentration of each test agent was 250 μg/ml. Tests were repeated five times in separate assays.

Hydroxyapatite discs were incubated in 20% pooled human saliva for 2 hours at room temperature. An amount of 10 ml of pooled human saliva was collected and combined with plaque inoculum scraped from the tooth surface of human volunteers. The inoculum was added to the 20% pooled saliva at a ratio of 1:3 (v/v) and 1.33 ml of the resulting suspension was combined with 2.0 ml artificial saliva (Pratten et al., 1998) and 0.175 ml of the appropriate test agent (Myrtus communis, Uncaria tormentosa, Orthosiphon spicatus, parsley or eucalyptus) at a concentration of 5 mg/ml in sterile water or water (as a negative control to which each test agent was compared). Parsley and eucalyptus were used as positive controls, as they are each well known natural ingredients in oral health products due to their positive effect on oral health.

Triplicate aliquots of each solution (1 ml) were placed in individual wells of a sterile 24 well plate with a single saliva coated hydroxyapatite disc. The discs were incubated for 1 hour at 37° C. in anaerobic conditions (10% H₂, 10% CO₂, 80% N₂), allowing the growth of obligate anaerobes that are found in the sub-gingival recesses associated with periodontitis. This was followed by 24 hours incubation at 37° C. in aerobic conditions.

Biofilms were dispersed, serially diluted and then plated onto CBA (+hemin, menadione) and incubated anaerobically or onto BHY and incubated aerobically. Colonies were counted after 24-48 hours. The results are shown in FIG. 1, where it can be seen that Myrtle (Myrtus communis) inhibited the numbers of facultative anaerobic bacteria in human plaque biofilms in vitro compared to untreated (water) control. Surprisingly, myrtle was more effective at reducing levels of these organisms than parsley and eucalyptus, known oral health promoters.

Fastidious anaerobe numbers were also counted, and were also seen to be reduced compared to untreated controls, as shown in FIG. 2. It was also unexpectedly found that myrtle performed better than parsley and eucalyptus in inhibiting fastidious anaerobes.

Myrtle leaf powder was also tested for inhibition of Peptostreptococcus stomatis growth in artificial saliva under plaque biofilm assay conditions described above (final concentration of the agents was 0.25 mg/ml). Colonies were counted after 24 hours growth in anaerobic cabinet.

Myrtle leaf treatment substantially reduced bacterial numbers in Peptostreptococcus biofilms compared to both untreated controls and those treated with eucalyptus leaf powder (FIG. 3). Peptostreptococcus are pathogenic bacteria, known to be associated with gingivitis, periodontitis and oral health problems.

### Example 8

Various product applications require survival of the raw material activity following exposure to temperatures up to 120° C. To test this, the raw myrtle leaf was heated to 120° C. for 10 minutes and its activity tested in the Plate Biofilm Assay compared with non heat-treated controls.

Heat treatment of Myrtus communis, as described above, does not affect its performance. Heat-treated Myrtus communis reduces biomass by 94.4%, compared to 97.7% in the unheated control. Protease is completely inhibited (100%) in both the heat-treated and non-heated control.

### Example 9

To assess product acceptance, myrtle leaf was included in a 25 g chew format at a level of 3% and fed to miniature schnauzers, cocker spaniels and Labradors in a crossover study with three other chew types. A chew was given once per day for 4 days and a washout period of 3 days was allowed before commencing the next feeding phase. When compared with the standard chew containing no myrtle, acceptance of the myrtle chew was similar in all dogs.

### Example 10

To assess the efficacy of myrtle for maintenance and improvement of oral health in companion animals myrtle leaf was included in a chew format at a level of 2.65% and fed to miniature schnauzers (17 g chew), cocker spaniels (25 g chew) and Labradors (40 g chew). The effect of the myrtle composition on oral health compared to that resulting from the standard chew, a second dental chew and to a dry kibble base diet was assessed. Thirty-two healthy adult dogs were assigned to one of 4 groups with a total of twelve Labrador Retrievers, twelve Cocker Spaniels and 8 Miniature Schnauzers. Animals were randomly assigned to groups within weighted blocks to ensure breed, sex and approximate age matching.

Animals lived in pairs in environmentally enriched two roomed housing with 24 h access to the outside and free access to exercise paddocks during daylight hours. Full animal welfare considerations were in place. The study was approved by the WALTHAM Centre for Pet Nutrition ethical review committee, in accordance with the UK Home Office Animals (Scientific Procedures) Act 1986. Dogs were socialized and walked daily and fresh water was available at all times. The animals were fed once daily at energy levels (calorific values) that were required in order to maintain bodyweight

The study utilised a four phase Latin square design with repeated measures. In this clean tooth model, the dogs were given a dental scale and polish at day 1 and received a standard commercial dry kibble diet and daily tooth brushing for two weeks (baseline phase) to reduce gingivitis to baseline levels. Gingivitis scores and removal of any accumulated dental deposits was then undertaken through a second dental scale and polish, following which animals received the same commercial dry kibble base diet plus test product for a five week period prior to repeated gingivitis scoring as well as measurement of plaque and calculus deposits. Group 1 (control animals) were maintained on the base diet only; group 2 in addition to base diet received a daily standard dental chew; group 3 received the same dental chew with 2.65% Myrtle leaf daily and group 4 received an alternative chew format not containing the active ingredient (data not shown for alternative chew format).

Following the end of phase 1 as described above each group transferred to the next dietary regime and repeated measures were taken in each subsequent phase until all of the dogs had received all of the diets. Gingivitis, plaque and calculus scores were assessed using the modified Logan & Boyce technique (Hennet et al., 2006) at the beginning and completion of the 5-week test period.

The following teeth were used for assessments of oral health.

### Maxilla: 13 (103,203), C (104,204), P2 (106,206), P3 (107,207), P4 (108,208), and M1 (109,209).

### Mandible: C (304,404), P2 (306,406), P3 (307,407), P4 (308,408), and M1 (309, 409).

Gingivitis was measured along the buccal surface at the gingival sulcus. The gingiva were divided into thirds (mesial, buccal and distal) and a score was given to each third. Tooth scores were calculated as the mean score of the three sections and total scores as the mean of all of the teeth assessed.

### Criteria

0—No gingivitis, pink (or pigmented) healthy gingiva no inflammation no bleeding on probing
1—Very mild gingivitis (red, swollen but no bleed on probing)
2—Mild gingivitis (red, swollen and delayed bleeding on probing)
3—Moderate gingivitis (red, swollen and immediate bleeding on probing)
4—Severe gingivitis (ulceration, spontaneous haemorrhage, profuse bleeding on probing)

Plaque was disclosed on the buccal surface of the teeth by applying an undiluted disclosing solution (Erythrosin) and immediately rinsing with water. Each of the scored teeth was assessed for coronal and gingival plaque levels according to Hennet et al. (2006). The two halves of the tooth crown (coronal and gingival) were successively assessed for plaque coverage and thickness was assessed on the uncovered part using a dye reference solution colour palette for the thickness assessment. The shade that is closest to that on the disclosed surface was designated as the thickness score. Scores on both the coronal and gingival sections were totalled to give a total tooth score. The means of all tooth scores provided the total mouth score.

Calculus was air-dried and a dental probe was used to gently verify the visual appearance of coverage and thickness. A coverage and thickness score was given for the gingival and coverage and thickness scores for gingival and coronal areas of the tooth were multiplied to give a total tooth score, mouth scores were calculated as for plaque coverage.

In addition to the clinical oral health assessments a supra-gingival plaque sample was scraped from the teeth of each dog during week 2 of the test phase. This was followed by thoroughly tooth brushing each dog to ensure any remaining dental deposits were removed.

Analyses were undertaken on the response variables plaque, gingivitis and calculus using a general linear model (GLM) to test for treatment, phase and sequence effects. Significance levels were reported along with estimates of treatment effects. Data were coded in Excel workbooks and analysed using proprietary statistical software routines (Minitab Verion 14).

### Results

The dental chews containing myrtle leaf powder significantly (P=<0.05) reduced mean gingivitis levels compared to base diet while the standard chew did not show significant reductions compared to base diet. Dogs being fed dental chews containing myrtle resulted in a mean gingivitis score below those observed at baseline following two weeks tooth brushing.

Mean plaque (P=<0.1) and calculus (P=<0.05) scores were reduced compared to standard diet but were slightly higher than those observed for dogs receiving the standard dental chew.

TABLE 2
Effect of the three dietary routines on clinical measures of oral health

| | Mean | | | |
|---|---|---|---|---|
| | Gingivitis | Mean Gingivitis | | |
| | Score | Score | Mean | Mean |
| | (end of test | (end of test phase | Plaque | Calculus |
| Diet | phase) | minus baseline ) | Score | Score |
| Standard Diet | 1.31 | 0.12 | 9.08 | 1.12 |
| Standard diet | 1.24 | 0.01 | 8.04 | 0.72 |
| and dental | | | | |
| chew | | | | |
| Standard diet | 1.20 | −0.01 | 8.27 | 0.79 |
| and dental | | | | |
| chew with | | | | |
| Myrtle | | | | |

### Example 11

An embodiment of the food product of the invention can be prepared by coextrusion. The food product is suitable for a dog. The outer portion and inner portion are as set out below.

Such a product may comprise, in approximate amounts, about 70% starch, about 4% palatants, about 5% water and the remainder being stabilisers, binders and preservatives in the outer portion in the form of a tough shell. This inner portion may comprise about 65% starch, about 5% palatants, about 6% water, about 4% myrtle, and the remainder being stabilisers, binders and preservatives.

The food product has an asymmetric triangular cross section. The edges formed by the corners of the asymmetric triangle twist through approximately 120° along the length of the product. This makes the product more difficult for the dog to chew, meaning that it will come into contact with a greater number of teeth.

The inner component is in the form of an aerated compressible starch matrix, with a very low protein content. This ensures high functionality of the myrtle in the inner portion. The outer portion contains no myrtle. Other active ingredients are included to enhance the oral care benefits of myrtle.

### Example 12

An alternative embodiment of the food product of the invention was prepared for a dog.

The composition of the inner and outer portions are as set out below.

Such a product may comprise, in approximate amounts, as the outer portion, about 80% starch, about 4% palatants, about 1% myrtle and about 1.5% hemp, the remainder being stabilisers, binders and preservatives and optionally colouring (for example marigold meal) in an amount up to 10%. The inner portion of such a product may comprise, in approximate amounts, about 75% fructose/glucose syrup, about 18% starch and about 8% myrtle. Such an inner portion has a gel-like texture.

The product is shaped as in FIG. 4. The food product has an approximate star shaped cross section. The edges formed by the parts of the star twist through 90° along the length of the product resulting in prolonged chewing/biting of the product. The inner component contains approximately 60% of the total myrtle and contains no protein for increased functionality of the myrtle. The inner portion is in the form of a gel. Further active ingredients are included in the outer portion for increased effectiveness.

### REFERENCES

Hennet P, Servet E, Salesse H, Soulard Y: Evaluation of the Logan and Boyce Plaque Index for the Study of Dental Plaque Accumulation in Dogs. Res Vet Sci, 80, 175-180, 2006.
Pratten, J., Smith, A. W. and Wilson, M. (1998) Response of single species biofilms and microcosm dental plaques to pulsing with chlorhexidine. J Antimicrob Chem 42, 453-459.

1. A food product comprising an outer portion and an inner portion, wherein the inner portion comprises myrtle and has a moisture content of from about 10% to about 25%.

2. The food product according to claim 1, wherein said outer portion is harder than said inner portion.

3. The food product according to claim 1, wherein said food product is in the form of a filled tube.

4. The food product according to claim 1, wherein said inner portion is in the form of a paste or a gel.

5. The food product according to claim 1, wherein said inner portion is a starch matrix.

6. The food product according to claim 1, wherein said outer portion comprises myrtle.

7. The food product according to claim 1, wherein said myrtle is present in said inner portion in an amount of from 1% to 10% by weight of said total product.

8. The food product according to claim 1, wherein the myrtle is present in the inner portion at an amount of about 4% by weight of the inner portion.

9. The food product according to claim 1, wherein said food product is a pet food product.

10. The food product according to claim 1, wherein said food product is a dog food product.

11. The food product according to claim 1, for use in the maintenance or improvement of oral health in an animal.

12. The food product according to claim 1, for use in the prevention or treatment of gingivitis in an animal.

13. The food product according to claim 1, for use in reducing tartar and/or dental plaque in an animal.

14. The food product according to claim 1, wherein the said myrtle is Myrtus communis.

15. The food product according to claim 1, wherein the animal is a cat, a dog or a human.

16. A method of maintaining or improving oral health in an animal comprising the step of administering to the animal an effective amount of the food product according to claim 1.

17. A method of making a food product according to claim 1 comprising the steps of mixing the ingredients that form the outer portion, mixing the ingredients that form the inner portion, and co-extruding the outer portion and inner portion to form a food product according to claim 1.

18-20. (canceled)
